(19) United States
(12) Patent Application Publication
(10) Pub. No.: US 2009/0202989 A1
(43) Pub. Date: Aug. 13, 2009

(54) EGFR AND KRAS MUTATIONS

(76) Inventor: Kenneth J. Hillan, San Francisco, CA (US)

Correspondence Address: JONES DAY, 222 EAST 41ST ST, NEW YORK, NY, 10017, US

(21) Appl. No.: 11/915,830

(22) PCT Filed: Jun. 13, 2006
(86) PCT No.: PCT/US06/23230

§ 371 Date: Nov. 28, 2007

(30) Foreign Application Priority Data
Jun. 28, 2006 (US) 60695174

(51) Int. Cl. C12Q 1/68; C12Q 1/48

(52) U.S. Cl. 435/6; 435/15

(57) ABSTRACT

The present invention relates to mutations in Epidermal Growth Factor Receptor (EGFR) and KRAS and methods of detecting such mutations as well as prognostic methods method for identifying a tumors that are susceptible to anticancer therapy such as chemotherapy and/or kinase inhibitor treatment. The methods involve determining the presence of a mutated EGFR gene or mutated EGFR protein and/or mutated KRAS gene or protein in a tumor sample.

### FIELD OF THE INVENTION

The present invention relates to cancer diagnostics and therapies and in particular to the detection of mutations that are diagnostic and/or prognostic.

### BACKGROUND OF THE INVENTION

Epidermal Growth Factor Receptor (EGFR) is a member of the type 1 tyrosine kinase family of growth factor receptors, which play critical roles in cellular growth, differentiation, and survival. Activation of these receptors typically occurs via specific ligand binding, resulting in hetero- or homodimerization between receptor family members, with subsequent autophosphorylation of the tyrosine kinase domain. This activation triggers a cascade of intracellular signaling pathways involved in both cellular proliferation (the ras/raf/MAP kinase pathway) and survival (the PI3 kinase/Akt pathway). Members of this family, including EGFR and HER2, have been directly implicated in cellular transformation.

A number of human malignancies are associated with aberrant or overexpression of EGFR and/or overexpression of its specific ligands e.g. transforming growth factor α (Gullick, Br Med Bull 1991, 47:87-98; Modijtahedi and Dean, Int J Oncol 1994, 4:277-96; Salomon et al., Crit Rev Oncol Hematol 1995;19:183-232). EGFR overexpression has been associated with an adverse prognosis in a number of human cancers, including NSCLC. In some instances, overexpression of tumor EGFR has been correlated with both chemoresistance and a poor prognosis (Lei et al., Anticancer Res 1999;19:221-8; Veale et al., Br J Cancer 1993;68:162-5). These observations suggest that agents that effectively inhibit EGFR receptor activation and subsequent downstream signaling may have clinical activity in a variety of human cancers, including NSCLC.

Tarceva™ (also known as erlotinib; OSI-774), a quinazoline, is an orally active, potent, selective inhibitor of EGFR tyrosine kinase. Erlotinib inhibits human EGFR tyrosine kinase with an IC₅₀ of 2 nM (0.786 mg/mL) in an in vitro enzyme assay. This inhibition is selective for EGFR tyrosine kinase, results in cell cycle arrest at G₁, and is reversible. Oral administration of erlotinib in mice has demonstrated a >70% reduction in EGFR autophosphorylation in human xenografts and marked growth inhibition of HN5 and A431 xenografts in nude mice has been demonstrated. In addition to single-agent activity in in vivo assay systems, erlotinib has been evaluated in combination with a number of chemotherapy agents to determine possible interactions. There was an additive interaction between erlotinib and paclitaxel, cisplatin, gemcitabine, and doxorubicin.

Lung cancer represents the leading cause of cancer-related mortality for both men and women in the United States. In 2000, it was estimated that 164,000 new cases would be diagnosed and 157,000 patients would die from this disease (Greenlee et al., CA Cancer J Clin 2001, 51:15-36). Approximately 75% of these patients would have had non-small cell histologies, with the majority presenting with inoperable Stage IIIB or Stage IV disease. For those patients with more limited disease at presentation (Stages I-IIIA), relapse following standard surgical therapy, with or without adjuvant or neoadjuvant chemo- and/or radiotherapy, is common. These findings result in an overall 5-year survival in non-small cell lung cancer (NSCLC) of ˜12% and serve to emphasize the unmet medical need in this disease.

The platinum compound cisplatin was the first chemotherapy agent to show clinical benefit in the management of locally advanced or metastatic NSCLC. Randomized clinical trials demonstrated improved response rates, quality of life, and survival compared with the best supportive care (Rapp et al. 1988). However, the magnitude of this improvement was modest-measured in weeks. Subsequently, a number of newer chemotherapy agents have been evaluated as single agents and in combination with the platinum salts in the first-line setting. The conclusion from these studies is that modern “doublet” chemotherapy appears to achieve response rates of 15%-20%, median time to disease progression of 3-4 months, and median survival of 7-8 months. The modest improvements in efficacy with combination therapies over the results obtained with cisplatin have established these therapies as a standard of care for patients with advanced NSCLC and an acceptable performance status (Non-Small Cell Lung Cancer Cooperative Group, Br Med J 1995, 311:899-909; American Society of Clinical Oncology, J Clin Oncol 1997, 15:2996-3018; Breathnach et al., J Clin Oncol 2001;19:1734-42).

### SUMMARY OF THE INVENTION

According to an aspect of the invention there is provided a method for identifying a tumor in a human subject that is susceptible to treatment comprising determining the presence of a mutated EGFR gene or mutated EGFR protein in a sample of said tumor wherein said mutation is located in exons 18-21 of EGFR whereby the presence of a mutated EGFR gene or mutated EGFR protein indicates the tumor is susceptible to treatment.

An another aspect of the invention there is provided a method of treating a tumor in a mammal comprising identifying the presence of an EGFR mutation in said tumor and treating said mammal with an anticancer agent.

In another aspect of the invention there is provided method of identifying an EGFR mutation in a sample comprising contacting nucleic acid from said sample with a probe that is capable of specifically hybridizing to nucleic acid encoding a mutated EGFR protein, or fragment thereof incorporating a mutation, and detecting the hybridization.

In another aspect of the invention there is provided nucleic acid probes capable of specifically hybridizing to nucleic acid encoding a mutated EGFR protein or fragment thereof incorporating a mutation.

In another aspect of the invention there is provided a method of detecting a mutated EGFR gene in a sample comprising amplifying from said sample nucleic acid corresponding to the kinase domain of said EGFR gene, or a fragment thereof suspected of containing a mutation, and comparing the electrophoretic mobility of the amplified nucleic acid to the electrophoretic mobility of corresponding wild-type EGFR gene or fragment thereof.

In another aspect of the invention there is provided a method for identifying a tumor in a human subject that is susceptible to treatment with an EGFR inhibitor comprising (i) determining the presence of a wild-type KRAS protein or gene in a sample of said tumor whereby the presence of a wild-type KRAS protein or gene indicates that the tumor is susceptible to treatment with an EGFR inhibitor or (ii) determining the presence of a mutated KRAS protein or gene in a sample of said tumor whereby the absence of a mutated KRAS protein or gene indicates that the tumor is susceptible to treatment with an EGFR inhibitor.

### BRIEF DESCRIPTION OF THE DRAWINGS

FIG. 1 illustrates the amino acid sequence of wild-type EGFR1 (SEQ ID NO: 1) in which the signal sequence is residues 1-24, the extracellular domain includes residues 24-645, the transmembrane domain includes residues 646-668, and the cytoplasmic domain includes residues 669-1210. The tyrosine kinase domain region is residues 718-964, and the threonine phosphorylation site is residue 678.

FIG. 2a through 2d is the cDNA sequence (SEQ ID NO: 2) of wild-type EGFR in which exon 18 corresponds to nucleotides 2308-2430; exon 19 corresponds to nucleotides 2431-2529; exon 20 corresponds to nucleotides 2530-2715 and exon 21 corresponds to 2716-2871.

FIG. 3 is a graphical representation of extracellular (top) and intracellular (bottom) regions of EGFR.

FIG. 4 is a Kaplan-Meier curve showing time to progression of patients having NSCLC tumors expressing wild-type EGFR (solid line) and mutant EGFR (dashed line).

FIG. 5 is a Kaplan-Meier curve showing survival of patients having NSCLC tumors expressing wild-type EGFR (solid line) and mutant EGFR (dashed line).

FIG. 6 is an autoradiograph illustrating inhibition of autophosphorylation of wild-type EGFR, and mutant EGFR (L858R and del746-752) with varying concentrations of erlotinib in transiently transfected COS7 cells.

FIG. 7 is a graph showing inhibition of autophosphorylation of wild-type EGFR and mutant EGFR (L858R and del746-752) with varying concentrations of erlotinib in transiently transfected COS7 cells.

FIG. 8 illustrates mutations in exons 18 and 19 of EGFR gene and protein sequences. Amino acid and nucleotide changes, and insertions are in bold, underlined font while deletions are shown as dashes (-).

FIG. 9 illustrates mutations in exons 20 and 21 of EGFR gene and protein sequences. Amino acid and nucleotide changes, and insertions are in bold, underlined font while deletions are shown as dashes (-).

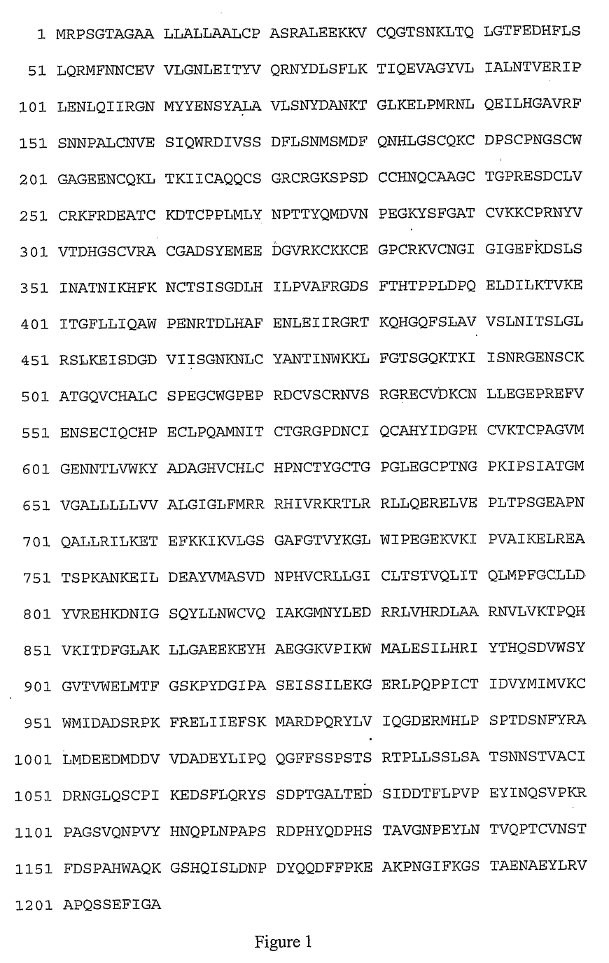

### DETAILED DESCRIPTION OF THE PREFERRED EMBODIMENTS

It is a discovery of the present invention that mutational events associated with tumorigenesis occur in Epidermal Growth Factor Receptor (EGFR). Although it was previously known that aberrant EGFR activity was associated with various cancers, it was unknown that mutations in the EGFR kinase domain region (KDR) existed that caused aberrant signaling activity associated with some cancers. Surprisingly patients. suffering from tumors having EGFR KDR mutations have a better prognosis than those with wild-type EGFR. The KDR mutations of the EGFR gene can involve rearrangements such as insertions and deletions as well as point mutations.

Samples from approximately 250 patients who participated a randomized, double-blinded phase III clinical trial referred to as Tribute were sequenced for mutations occurring in exons 18-21 of EGFR. Tribute studied 1,079 patients at approximately 150 centers in the United States having histological confirmed NSCLC who had not received prior chemotherapy comparing erlotinib+chemotherapy (carboplatin/paclitaxel) with chemotherapy alone. Patients received paclitaxel (200 mg/m² 3 hour i.v. infusion) followed by carboplatin (AUC=6 mg/ml×minute infused over 15-30 minutes using Calvert formula) with or without erlotinib (100 mg/day p.o. escalated to 150 mg/day for tolerant patients). Tumor samples, formalin-fixed paraffin-embedded blocks or unstained slides, from approximately 250 patients collected from the Tribute trial were enriched for tumor cells by laser capture mircrodissection followed by DNA extraction. Exons 18-21 were amplified by nested PCR and bi-directional sequences were obtained from each PCR product using fluorescent dye-terminator chemistry. Mutations discovered from the sequencing are shown in table 1:

TABLE 1

| | | |
|---|---|---|
| protein mutation | nucleic acid mutation | exon |

| | | |
|---|---|---|
| G719A | 2402G > C | 18 |
| G719C | 2401G > T | 18 |
| G719S | 2401G > A | 18 |
| E746-R748 del | 2482-2490 del GGAATTAAGA | 19 |
| | (SEQ ID NO: 32) | |
| E746-A750 del | 2481-2495 del GGAATTAAGAGAAGC | 19 |
| | (SEQ ID NO: 33) | |
| E746-R748 del | 2482-2490 del GAATTAAGA | 19 |
| E749Q | 2491G > C | |
| A750P | 2494G > C | |
| L747-E749 del | 2485-2493 del TTAAGAGAA | 19 |
| A750P | 494G > C | |
| L747S | 2486-2503 del TAAGAGAAGCAACATCTC | 19 |
| R748-P753 del | (SEQ ID NO: 34) | |
| L747-S752 del | 2485-2502 del TTAAGAGAAGCAACATCT | 19 |
| E746V | 2483A > T | |
| | (SEQ ID NO: 35) | |
| L747-T751 del | 2486-2494del TAAGAGAAGCAA | 19 |
| ins S | (SEQ ID NO: 36) | |
| S752-I759 del | 2499-2522 del ATCTCCGAAAGCCAACAAGGAAAT | 19 |
| | (SEQ ID NO: 37) | |
| M766-A767 AI ins | 2544-2545 ins GCCATA | 20 |
| S768-V769 SVA ins | 2554-2555 ins CCAGCGTGG | 20 |
| | (2556C > T silent) | |
| L858R | 2819T > G | 21 |
| G719C | 2401G > T | 18 |
| S768I | 2549G > T | 20 |
| | {2607G > A SNP silent} | |
| G719C | 2401G > T | 18 |
| V765M | 2539G > A | 20 |
| S768I | 2549G > T | 20 |
| A755V | 2510C > T | 19 |
| L747S | 2486T > C | 19 |
| E746K | 2482G > A | 19 |
| P772-H773 V ins | 2561-2562 ins GGT | 20 |
| L858P | 2819T > C | 21 |
| L861Q | 2576T > A | 21 |
| P772-H773 NS ins | 2562-2563 ins AACTCC | 20 |
| H773Y | 2563C > T | |
| T790M | 2615C > T | 20 |
| L858R | 2819T > G | 21 |
| S784F | | 21 |
| L858R | | 21 |

ins = insertion
del = deletion

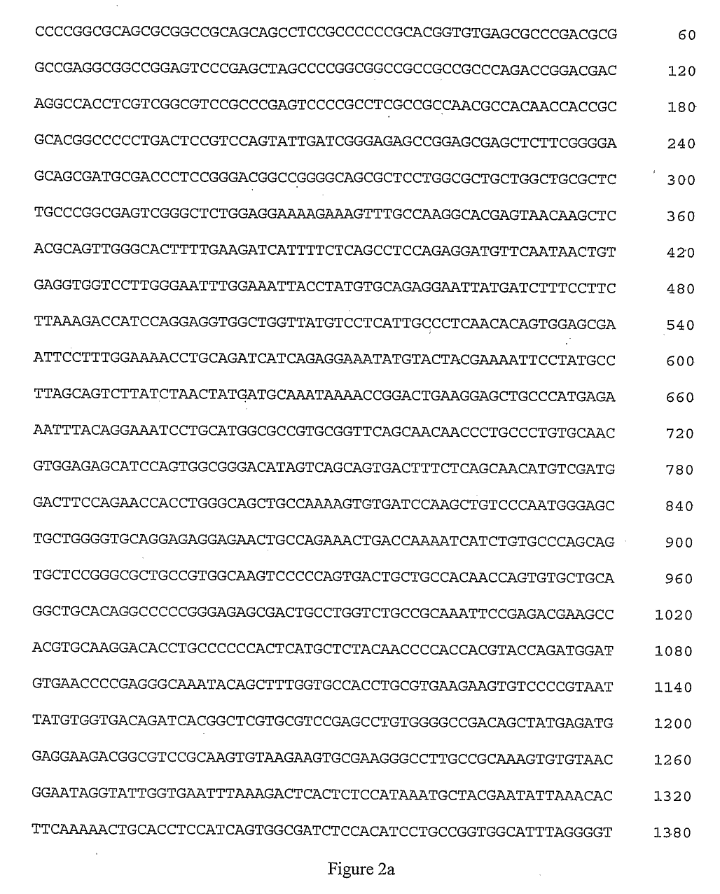

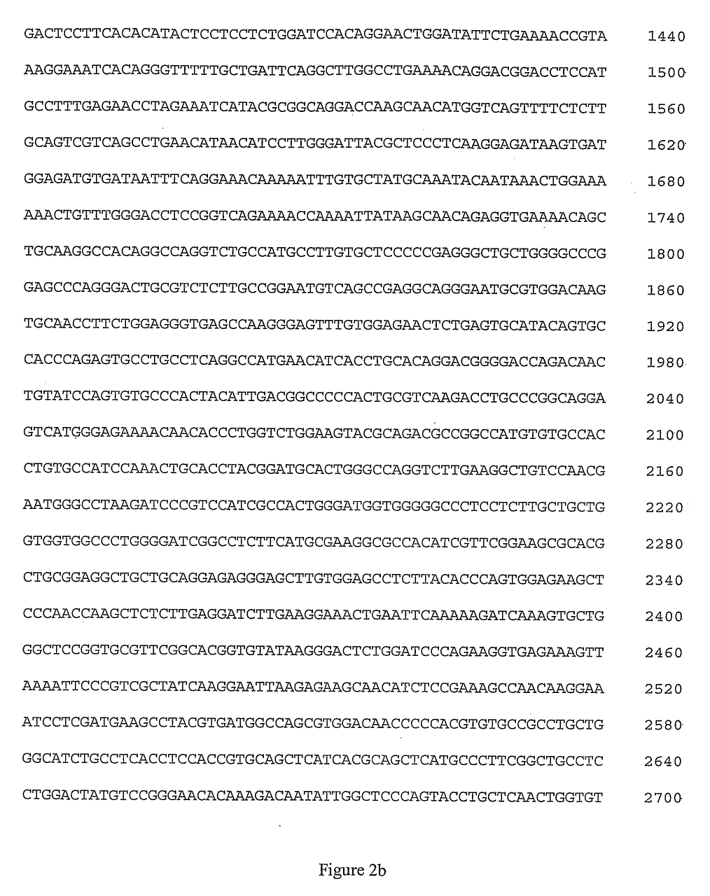

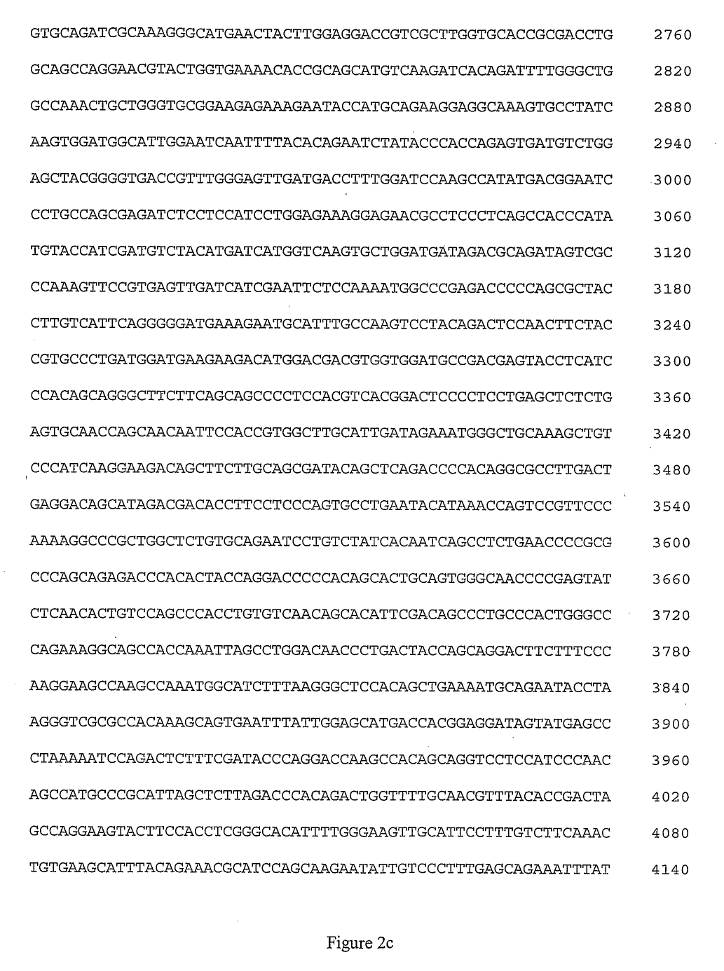

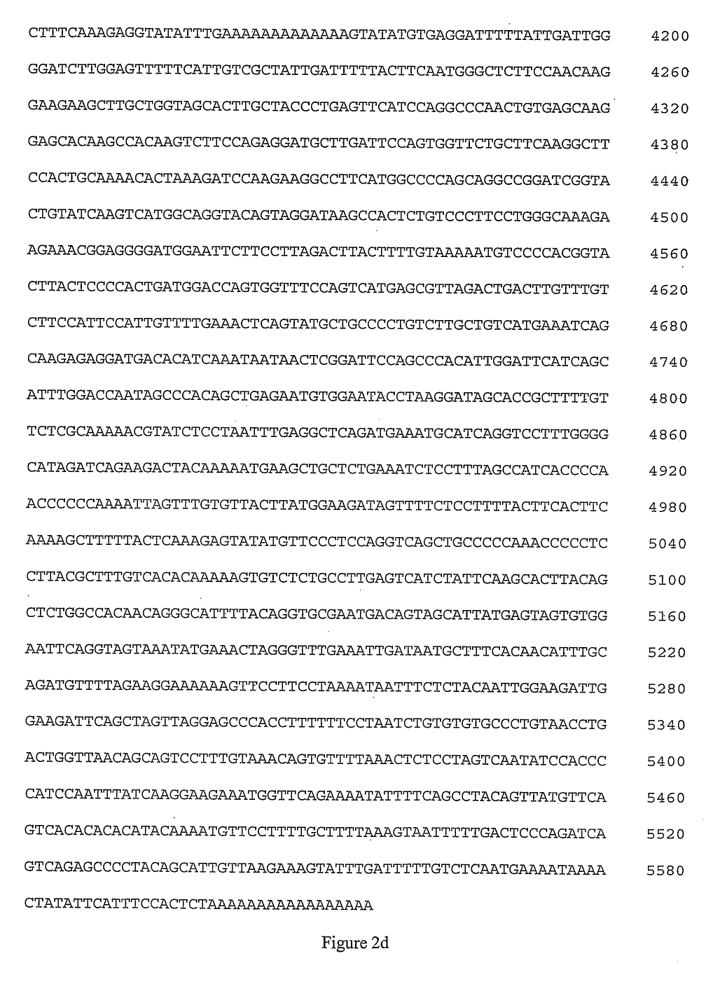

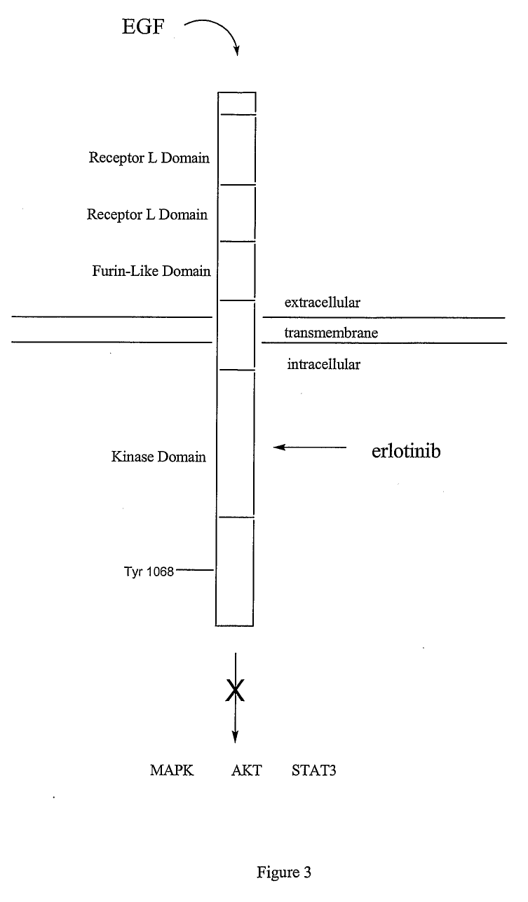

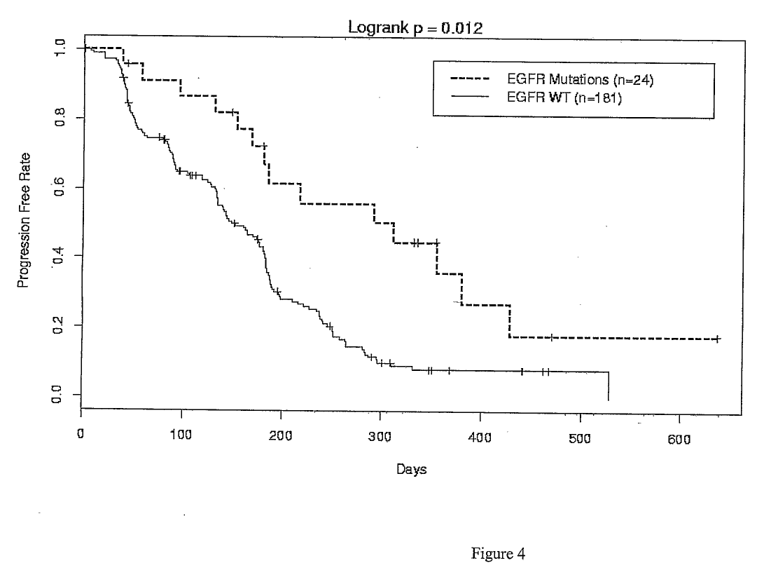

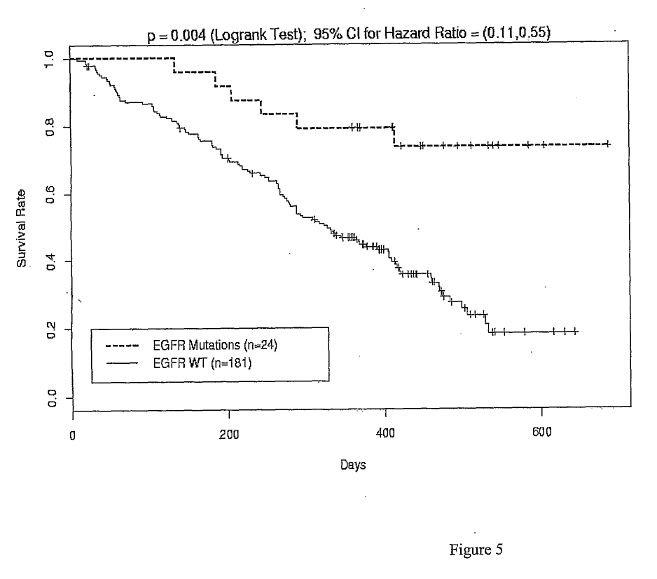

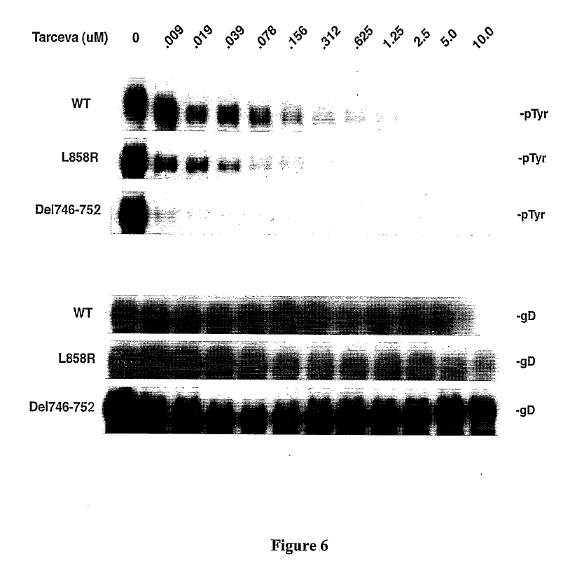

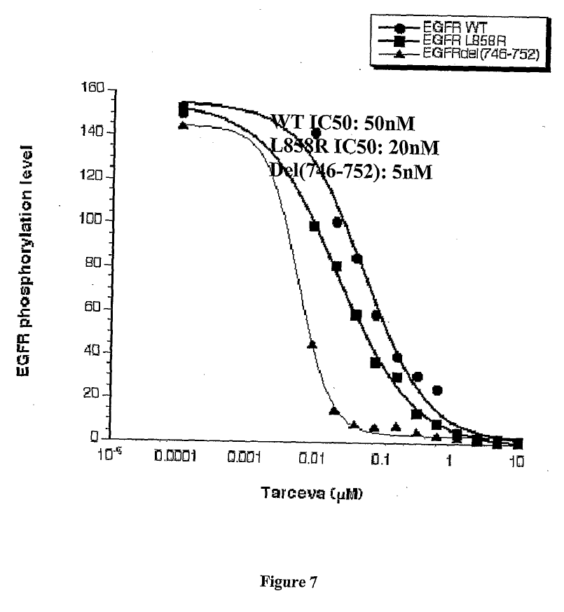

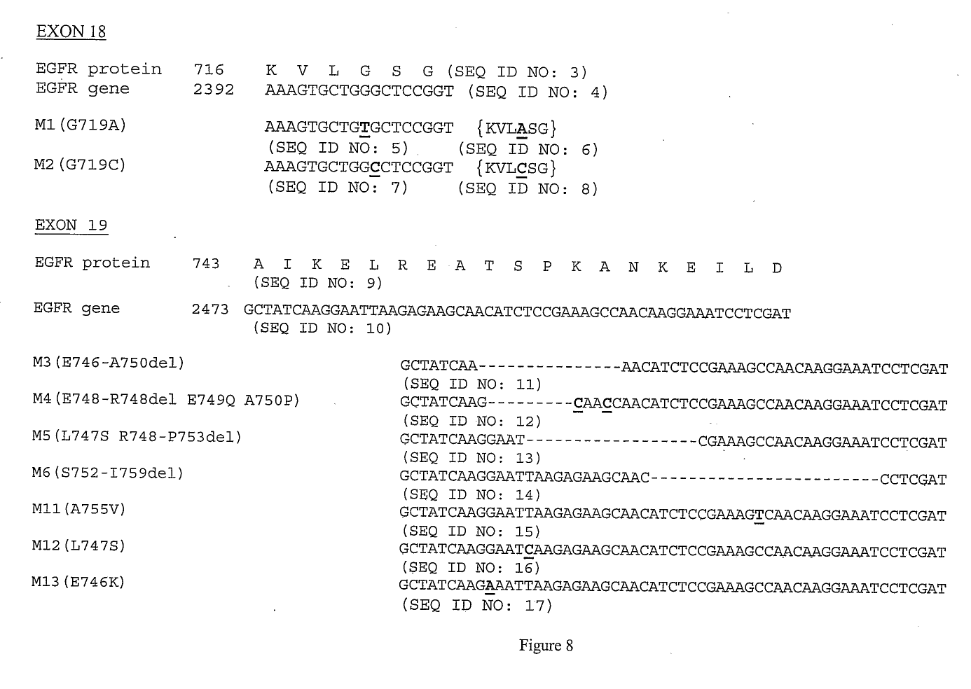

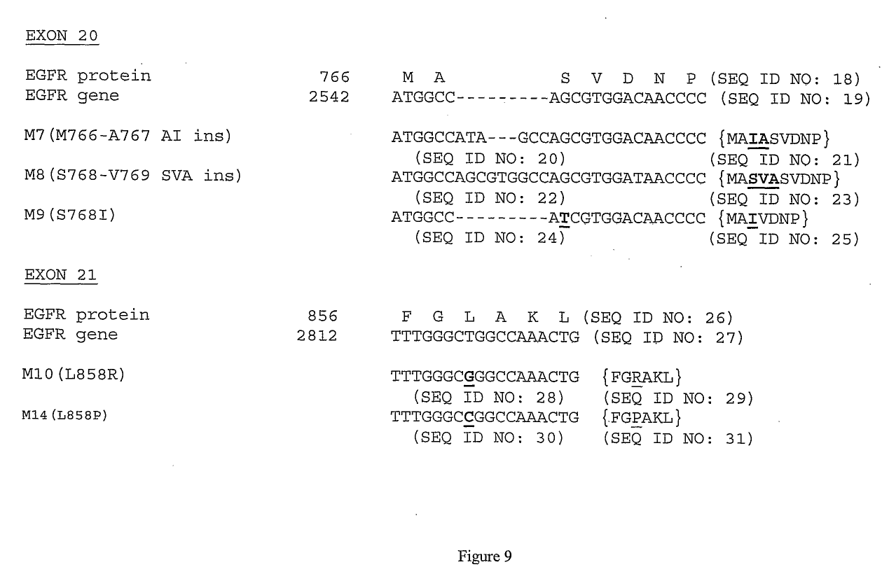

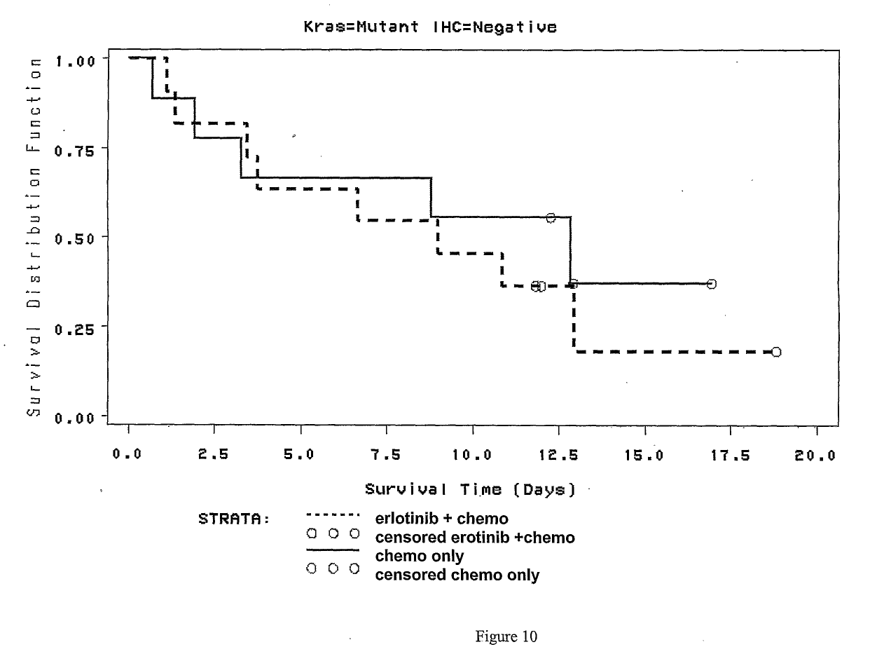

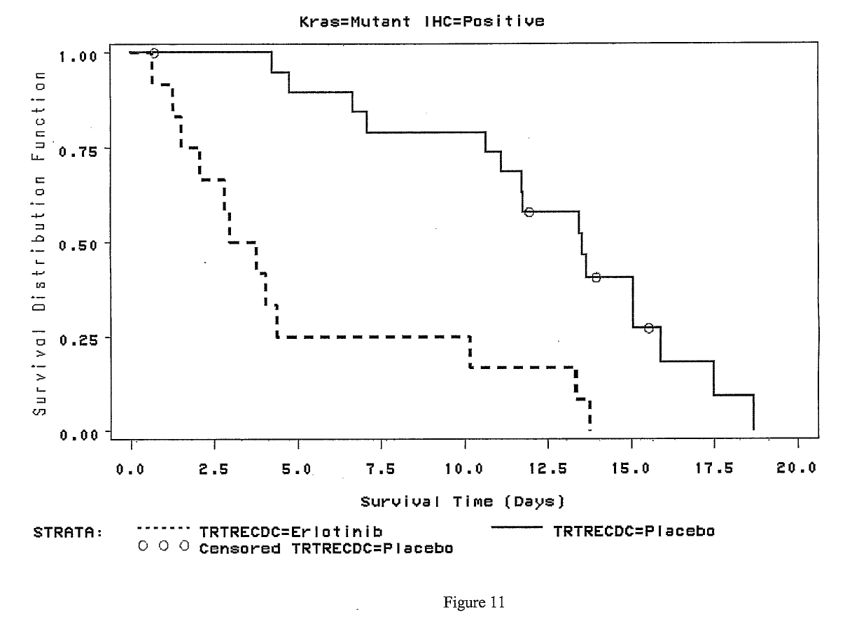

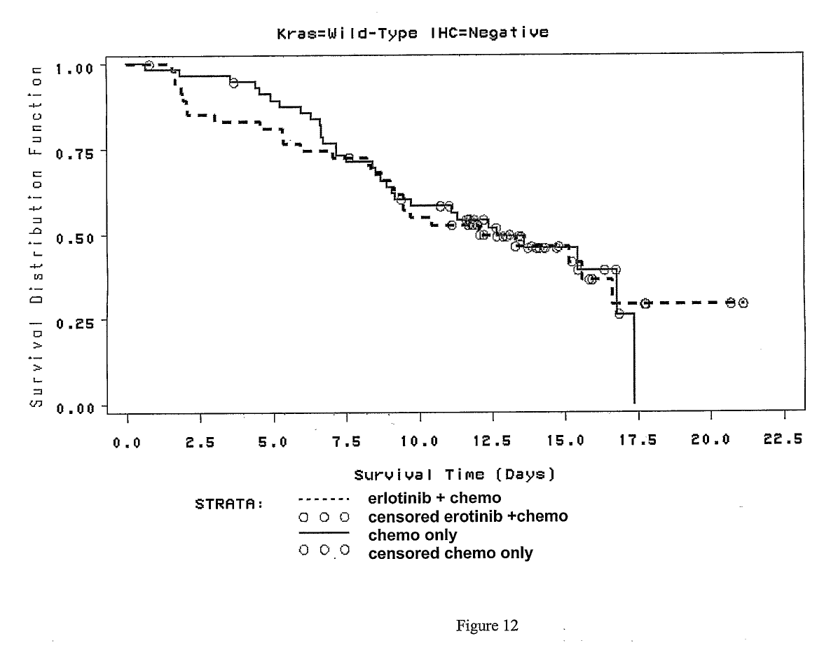

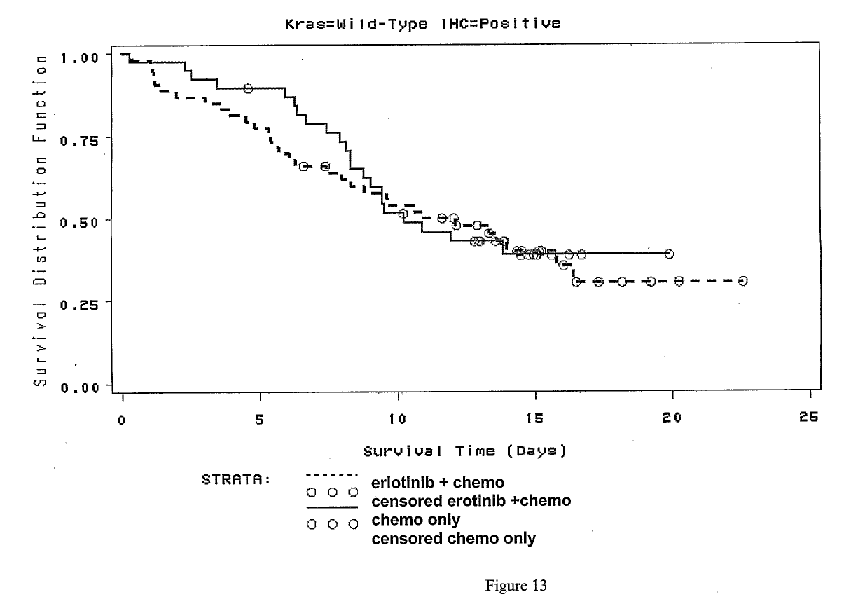

Nucleotide numbering for mutations is based on reference sequence shown in FIGS. 2a-2d.

Clinical outcome of patients having tumors with EGFR mutations and wild-type EGFR were analyzed according to response (complete+partial) benefit (response+stable disease) and progressive disease. Lesions were evaluated using Response Evaluation Criteria in Solid Tumors (RECIST) criteria whereby “complete response” (CR) is defined as the disappearance of all target lesions; “partial response” (PR) is defined as at least a 30% decrease in the sum of the longest diameter of target lesions, taking as reference the baseline sum longest diameter; “progressive disease” (PD) is defined as at least a 20% increase in the sum of the longest diameter of target lesions, taking as reference the smallest sum longest diameter recorded since the treatment started or the appearance of one or more new lesions; and “stable disease” (SD) is defined as neither sufficient shrinkage to qualify for partial response nor sufficient increase to qualify for progressive disease, taking as reference the smallest sum longest diameter since the treatment started.

Results of the analysis are summarized in table 2.

TABLE 2

| | |
|---|---|
| Mutant EGFR | Wild-Type EGFR |
| n = 24 | n = 181 |

Response/Benefit Rate

| | | | | |
|---|---|---|---|---|
| response (CR + PR) | 11 | 46% | 46 | 25% |
| benefit (CR + PR + SD) | 18 | 75% | 105 | 58% |
| SD | 7 | 29% | 59 | 33% |
| PD | 6 | 25% | 76 | 42% |

Survival (days)

| | | |
|---|---|---|
| median | 435 | 309 |
| range | 133-687 | 9-643 |

CR = complete response; PR = partial response; SD = stable disease; PD = progressing disease

Analysis of clinical outcome revealed that patients with tumors expressing a mutation in exons 18-21 of EGFR have better prognosis than those with tumors expressing wild-type EGFR. Mutant EGFR patients exhibited greater response rate, benefit rate and survival when treated with chemotherapy or chemotherapy plus erlotinib. These results are useful for predicting outcome such that patients whose tumors have EGFR mutations in any or all of exons 18 through 21 have more favorable prognosis than patients whose tumors do not have such mutations.

Accordingly, the present invention provides a method for determining the prognosis of a patient having a tumor comprising determining in a sample of said tumor the presence or absence of one or more EGFR mutations in exons 18-21 (or the amino acid sequence corresponding to exons 18-21) whereby the presence of said one or more EGFR mutation indicates better prognosis compared to the absence of said one or more EGFR mutation. By “prognosis” is meant response and/or benefit and/or survival. By “EGFR mutations” means an amino acid or nucleic acid sequence that differs from wild-type EGFR protein or nucleic acid respectively found on one allele (heterozygous) or both alleles (homozygous) and may be somatic or germ line. In a particular embodiment said mutation is found in the kinase domain region (KDR) of EGFR. In another particular embodiment the mutation is an amino acid substitution, deletion or insertion as shown in table 1. In an embodiment the amino acid mutation is one or more of the following: G719A, E746K, L747S, E749Q, A750P, A755V, S768I, L858P, E746-R748 del, R748-P753 del, M766-A767 AI ins, and S768-V769 SVA ins. In another particular embodiment, the mutation is a nucleic acid point mutation, deletion or insertion as shown in table 1. In an embodiment, the nucleic acid mutation is one or more the following: 2402G>C; 2482G>A; 2486T>C; 2491G>C; 2494G>C; 2510C>T; 2549G>T; 2819T>C; 2482-2490 del; 2486-2503 del; 2544-2545 ins GCCATA; and 2554-2555 ins CCAGCGTGG.

EGFR exons 18-21 from an H1975 tumor cell line that exhibited resistance to treatment with erlotinib was sequenced and found to incorporate a mutation T790M in combination with an L858R mutation. Accordingly the present invention further provides a method for determining the prognosis of a patient having a tumor comprising determining in a sample of said tumor the presence or absence of the T790M EGFR mutation whereby the presence of said T790M EGFR mutation indicates poorer prognosis compared to the absence of said T790M EGFR mutation. Further, there is provided a method of identifying patients having a tumor that is less responsive to therapy of an EGFR inhibitor such as erlotinib or gefitinib, whether in combination with chemotherapy or not, comprising determining the presence or absence of a T790M EGFR mutation in the patient's tumor whereby the presence of said mutation indicates the patient will respond less to said therapy compared to a patient having a tumor that does not have said T790M EGFR mutation. Further, there is provided a method of identifying a tumor that is resistant to treatment with an EGFR inhibitor, such as a kinase domain binding inhibitor (for example erlotinib or gefitinib), whether in combination with chemotherapy or not, comprising determining the presence or absence of a T790M EGFR mutation in a sample of the tumor whereby the presence of said mutation indicates the tumor is resistant to said treatment. It is understood that determination of the mutation is at the protein level or nucleic acid level (genomic DNA or mRNA) and are accomplished using techniques such as those described herein. In a particular embodiment, said EGFR inhibitor competes with ATP at the EGFR kinase domain. In a particular embodiment the EGFR inhibitor is erlotinib.

In another aspect, there is provided a method of treating a patient having a tumor incorporating a T790M mutant EGFR protein or gene (or treating a tumor incorporating a T790M mutant EGFR protein or gene) comprising co-administering to said patient (or contacting said tumor with) a first compound that binds to and/or inhibits signaling of said T790M mutant EGFR in combination with a second compound that binds to and/or inhibits signaling of wild-type EGFR or EGFR incorporating an activating mutation. In a particular embodiment said activating mutation is one or more of those described in Table 1 (other than T790M). In a particular embodiment said first and second compounds are administered sequentially or concomitantly. In a particular embodiment said second compound is erlotinib.

In another aspect of the invention, there is provided a method of screening for compounds that inhibit signaling of a mutant EGFR protein that incorporates a T790M mutation, comprising contacting said mutant EGFR with a test compound in the presence of a phosphorylation substrate and ATP and detecting a change in the amount of phosphorylation of said substrate whereby a reduction of phosphorylation of said substrate compared to a control, or compared to phosphorylation of the substrate in the absence of the test compound, indicates said test compound is an inhibitor of mutant EGFR signaling. In an embodiment, said method is performed in vitro in the presence of a ligand for said mutant EGFR such as EGF or TGF-alpha.

In a particular embodiment the inhibitory activity of a test compound can be determined in vitro by the amount of inhibition of the phosphorylation of an exogenous substrate (e.g. Lys₃-Gastrin or polyGluTyr (4:1) random copolymer (I. Posner et. al., J. Biol. Chem. 267 (29), 20638-47 (1992)) on tyrosine by epidermal growth factor receptor kinase by a test compound relative to a control. Purified, soluble human T790M mutant EGFR (96 ng) is preincubated in a microfuge tube with EGF (2 μg/ml) in phosphorylation buffer+vanadate (PBV: 50 mM HEPES, pH 7.4; 125 mM NaCl; 24 mM MgCl₂; 100 μM sodium orthovanadate), in a total volume of 10 μl, for 20-30 minutes at room temperature. The test compound, dissolved in dimethylsulfoxide (DMSO), is diluted in PBV, and 10 μl is mixed with the mutant EGFR/EGF mix, and incubated for 10-30 minutes at 30° C. The phosphorylation reaction is initiated by addition of 20 μl ³³P-ATP/substrate mix (120 μM Lys₃-Gastrin (sequence in single letter code for amino acids, KKKGPWLEEEEEAYGWLDF - SEQ ID NO: 38), 50 mM Hepes pH 7.4, 40 μM ATP, 2 μCi γ-[³³P]-ATP) to the mutant EGFR/EGF mix and incubated for 20 minutes at room temperature. The reaction is stopped by addition of 10 μl stop solution (0.5 M EDTA, pH 8; 2 mM ATP) and 6 μl 2N HCl. The tubes are centrifuged at 14,000 RPM,. 4° C., for 10 minutes. 35 μl of supernatant from each tube is pipetted onto a 2.5 cm circle of Whatman P81 paper, bulk washed four times in 5% acetic acid, 1 liter per wash, and then air dried. This results in the binding of substrate to the paper with loss of free ATP on washing. The [³³P] incorporated is measured by liquid scintillation counting. Incorporation in the absence of substrate (e.g., lys₃-gastrin) is subtracted from all values as a background and percent inhibition is calculated relative to controls without test compound present. Such assays, carried out with a range of doses of test compounds, allow the determination of an approximate IC₅₀ value for the in vitro inhibition of T790M mutant EGFR kinase activity.

In another aspect of the invention there is provided a method for identifying a tumor in a human subject that is susceptible to treatment comprising determining the presence of a mutated EGFR gene or mutated EGFR protein in a sample of said tumor wherein said mutation is located in exons 18-21 of EGFR whereby the presence of a mutated EGFR gene or mutated EGFR protein indicates that the tumor is susceptible to treatment with an anticancer agent. In a particular embodiment the anticancer agent is a chemotherapeutic agent which may be a cytotoxic or cytostatic. Tumors include neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma. Particular tumors include those of the brain, liver, kidney, bladder, breast, gastric, ovarian, colorectal, prostate, pancreatic, breast, lung, vulval, thyroid, colorectal, oesophageal, hepatic carcinomas, sarcomas, glioblastomas, head and neck, leukemias and lymphoid malignancies.

Particular chemotherapeutic agents include, but are not limited to (i) antimetabolites, such as cytarabine, fludarabine, 5-fluoro-2′-deoxyuiridine, gemcitabine, hydroxyurea or methotrexate; (ii) DNA-fragmenting agents, such as bleomycin, (iii) DNA-crosslinking agents, such as chlorambucil, cisplatin, cyclophosphamide or nitrogen mustard; (iv) intercalating agents such as adriamycin (doxorubicin) or mitoxantrone; (v) protein synthesis inhibitors, such as L-asparaginase, cycloheximide, puromycin or diphtheria toxin; (Vi) topoisomerase I poisons, such as camptothecin or topotecan; (vii) topoisomerase II poisons, such as etoposide (VP-16) or teniposide; (viii) microtubule-directed agents, such as colcemid, colchicine, paclitaxel, vinblastine or vincristine; (ix) kinase inhibitors such as flavopiridol, staurosporin, STI571 (CPG 57148B) or UCN-01 (7-hydroxystaurosporine); (x) miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; (xi) hormones such as glucocorticoids or fenretinide; (xii) hormone antagonists, such as tamoxifen, finasteride or LHRH antagonists. In an embodiment, the chemotherapeutic compound is one or more of gemcitabine, cisplatin, doxorubicin, daunarubicin, paclitexel, taxotere and mitomycin C. In a particular embodiment the chemotherapeutic compound is one or more of gemcitabine, cisplatin and paclitaxel. In another embodiment the treatment is an inhibitor of EGFR. In an embodiment the EGFR inhibitor is an antibody such as Erbitutux™ (cetuximab, Imclone Systems Inc.) and ABX-EGF (panitumumab, Abgenix, Inc.). In another embodiment the EGFR inhibitor is a small molecule that competes with ATP such as Tarceva™ (erlotinib, OSI Pharmaceuticals), Iressa™ (gefitinib, Astra-Zeneca), tyrphostins described by Dvir, et al., J Cell Biol., 113:857-865 (1991); tricyclic pyrimidine compounds disclosed in U.S. Pat. No. 5,679,683; compound 6-(2,6-dichlorophenyl)-2-(4-(2-diethylaininoethoxy)phenylamino)-8-methyl-8H-pyrido(2,3-d)pyrimidin-7-one (known as PD166285) disclosed in Panek, et al., Journal of Pharmacology and Experimental Therapeutics 283, 1433-1444 (1997).

In another aspect of the invention there is provided a method of identifying an EGFR mutation in a sample comprising contacting nucleic acid from said sample with a nucleic acid probe that is capable of specifically hybridizing to nucleic acid encoding a mutated EGFR protein, or fragment thereof incorporating a mutation, and detecting said hybridization. In a particular embodiment said probe is detectably labeled such as with a radioisotope (³H, ³²P, ³³P etc), a fluorescent agent (rhodamine, fluorescene etc.) or a chromogenic agent. In a particular embodiment the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect said probes of the invention are provided in a kit for identifying EGFR mutations in a sample, said kit comprising an oligonucleotide that specifically hybridizes to or adjacent to a site of mutation in the EGFR gene. The kit may further comprise instructions for treating patients having tumors that contain EGFR mutations with an EGFR inhibitor based on the result of a hybridization test using the kit.

In another aspect of the invention there is provided a method of detecting a mutated EGFR gene in a sample comprising amplifying from said sample nucleic acid corresponding to the kinase domain of said EGFR gene, or exons 18-21, or a fragment thereof suspected of containing a mutation, and comparing the electrophoretic mobility of the amplified nucleic acid to the electrophoretic mobility of corresponding wild-type EGFR gene or fragment thereof. A difference in the mobility indicates the presence of a mutation in the amplified nucleic acid sequence. Electrophoretic mobility may be determined on polyacrylamide gel.

Alternatively, amplified EGFR gene or fragment nucleic acid may be analyzed for detection of mutations using Enzymatic Mutation Detection (EMD) (Del Tito et al, Clinical Chemistry 44:731-739, 1998). EMD uses the bacteriophage resolvase T₄ endonuclease VII, which scans along double-stranded DNA until it detects and cleaves structural distortions caused by base pair mismatches resulting from point mutations, insertions and deletions. Detection of two short fragments formed by resolvase cleavage, for example by gel eletrophoresis, indicates the presence of a mutation. Benefits of the EMD method are a single protocol to identify point mutations, deletions, and insertions assayed directly from PCR reactions eliminating the need for sample purification, shortening the hybridization time, and increasing the signal-to-noise ratio. Mixed samples containing up to a 20-fold excess of normal DNA and fragments up to 4 kb in size can been assayed. However, EMD scanning does not identify particular base changes that occur in mutation positive samples requiring additional sequencing procedures to identity of the mutation if necessary. CEL I enzyme can be used similarly to resolvase T₄ endonuclease VII as demonstrated in U.S. Pat. No. 5,869,245.

Another simple kit for detecting the EGFR mutations of the invention is a reverse hybridization test strip similar to Haemochromatosis StripAssay™ (Viennalabs http://www.bamburghmarrsh.com/pdf/4220.pdf) for detection of multiple mutations in HFE, TFR2 and FPN1 genes causing Haemochromatosis. Such an assay is based on sequence specific hybridisation following amplification by PCR. For single mutation assays, a microplate-based detection system may be applied, whereas for multi-mutation assays, teststrips may be used as “macro-arrays”. Kits may include ready-to use reagents for sample prep, amplification and mutation detection. Multiplex amplification protocols provide convenience and allow testing of samples with very limited volumes. Using the straightforward StripAssay format, testing for twenty and more mutations may be completed in less than five hours without costly equipment. DNA is isolated from a sample and the EGFR gene (or exons 18-21 or KDR or segments thereof) is amplified in vitro (e.g. PCR) and biotin-labeled, preferably in a single (“multiplex”) amplification reaction. The PCR products are the selectively hybridized to oligonucleotide probes (wild-type and mutant specific) immobilized on a solid support such as a test strip in which the probes are immobilized as parallel lines or bands. Bound biotinylated amplicons are detected using streptavidin-alkaline phosphatase and color substrates. Such an assay can detect all or any subset of the mutations in table 1. With respect to a particular mutant probe band one of three signaling patterns are possible: (i) a band only for wild-type probe which indicates normal EGFR (ii) bands for both wild-type and a mutant probe which indicates heterozygous genotype and (iii) band only for the mutant probe which indicates homozygous mutant EGFR genotype. Accordingly there is farther provides a method of detecting EGFR mutations of the invention comprising isolating nucleic acid from a sample, amplifying the EGFR gene, or fragment thereof (e.g. the KDR or exons 18-21 or smaller) such that the amplified nucleic acid comprises a ligand, contacting the amplified EGFR gene or fragment with a probe which comprises a detectable binding partner to the ligand and the probe is capable of specifically hybridizing to an EGFR mutation, and then detecting the hybridization of said probe to said amplified EGFR gene or fragment. In a particular embodiment the ligand is biotin and the binding partner is comprises avidin or streptavidin. In a particular embodiment the binding partner is steptavidin-alkaline which is detectable with color substrates. In a particular embodiment the probes are immobilized for example on a test strip wherein probes complementary to different mutations are separated from one another. Alternatively, the amplified nucleic acid is labeled with a radioisotope in which case the probe need not comprise a ligand.

The tumor samples were also analyzed for mutations in KRAS (as referred to as p21a). Particular mutations detected in exon 1 are: G12C; G12A; G12D; G12R; G12S; G12V; G13C; G13D which correlated with poor prognosis to chemotherapy as well as chemotherapy with erlotinib therapy. Accordingly, the invention further provides a method of identifying patients not responsive to therapy of an EGFR inhibitor such as erlotinib or erlotinib in combination with chemotherapy comprising determining the presence or absence of a KRAS mutation whereby the presence of said mutation indicates a patient will not respond to said therapy. Alternatively, there is provided a method for identifying a tumor in a human subject that is susceptible to treatment with an EGFR inhibitor comprising (i) determining the presence of a wild-type KRAS protein or gene in a sample of said tumor whereby the presence of a wild-type KRAS protein or gene indicates that the tumor is susceptible to treatment with an EGFR inhibitor or (ii) determining the presence of a mutated KRAS protein or gene in a sample of said tumor whereby the absence of a mutated KRAS protein or gene indicates that the tumor is susceptible to treatment with an EGFR inhibitor. In a particular embodiment the KRAS mutation is an activating mutation. In a particular embodiment the mutation is in exon 1 of KRAS. In another embodiment the KRAS mutation is at least one of G12C; G12A; G12D; G12R; G12S; G12V; G13C; G13D. Alternatively, individuals who have tumors which harbor mutant KRAS may be treated with EGFR inhibitors when also treated with a KRAS inhibitor either before, after or during treatment with the EGFR inhibitor. Methods for determining the presence of KRAS mutations are analogous to those used to identify EGFR mutations described in detail herein.

It was further observed that patients whose tumors were found to have present KRAS mutations and stained positively for EGFR by IHC had significantly shorter survival when treated with erlotinib in combination with chemotherapy compared to those patients treated with chemotherapy alone. The following tables 3 and 4 summarize these results. Accordingly there is provided a method of identifying a patient nonresponsive to treatment of an EGFR inhibitor such as erlotinib, either alone or in combination with a chemotherapeutic agent, comprising determining the presence or absence of a KRAS mutation and an EGFR in a tumor of said patient whereby the presence of both a KRAS mutation and an EGFR in said tumor indicates a patient will not respond to said EGFR inhibitor therapy either alone or in combination with chemotherapy. In this context “nonresponsive” means that a patient will not have a response according to RECIST criteria, or will have a reduced survival than a similar patient (having KRAS mutations and the presence of EGFR a in tumor) treated with chemotherapy alone. Said EGFR may be either mutant or wildtype EGFR and may be determined by any technique including but not limited to those described herein. KRAS mutations refers to mutations in the KRAS protein or nucleic acid and is detected using procedures analogous to those for detecting EGFR mutations. In an embodiment, the presence of EGFR is determined by immunohistochemistry (IHC). In another embodiment the presence of EGFR is determined by fluorescence in situ hybridization detection of increased levels of EGFR nucleic acid compared to a normal cell. In another embodiment, the EGFR is wildtype EGFR. In another embodiment, the EGFR is a mutant EGFR. In a particular embodiment, nonresponse is a lack of complete response (CR) according to RECIST criteria. In another embodiment, nonresponse is a lack of partial response (PR) according to RECIST criteria. In another embodiment, nonresponse is lack of stable disease (SD) according to RECIST criteria. In another embodiment, the nonresponse is a reduced survival period.

Alternatively, there is provided a method of determining whether a tumor will respond to treatment with an EGFR inhibitor, either alone or in combination with a chemotherapeutic agent, comprising determining in a sample of said tumor the presence of a mutant KRAS protein or nucleic acid and an EGFR, whereby the presence of both a mutant KRAS protein or nucleic acid and an EGFR indicates that the tumor will not respond to treatment with an EGFR inhibitor. In this context, “respond” means that the tumor will shrink in size or volume or the rate of increase in size or volume is reduced. In a particular embodiment, said treatment with an EGFR inhibitor is prior to, concurrent with, or subsequent to with treatment with chemotherapy.

TABLE 3
Kras Wild Type

| | |
|---|---|
| erlotinib + Chemo | Chemo Alone |

EGFR IHC−

| | | |
|---|---|---|
| n | 48 | 56 |
| Median OS (mo) (95% Cl) | 12.1 (9.0, 16.6) | 12.7 (9.1, 16.8) |

| | |
|---|---|
| Logrank P-value | 0.9557 |

EGFR IHC+

| | | |
|---|---|---|
| n | 53 | 38 |
| Median OS (mo) (95% Cl) | 12.1 (8.0, 16.6) | 10.3 (8.3, .) |

| | |
|---|---|
| Logrank P-value | 0.7892 |
| Hazard Ratio (95% Cl) | 1.1 (0.6, 1.9) |

TABLE 4
Kras Mutants

| | |
|---|---|
| erlotinib + Chemo | Chemo Alone |

EGFR IHC−

| | | |
|---|---|---|
| n | 11 | 9 |
| Median OS (mo) (95% Cl) | 9.0 (3.4, 12.9) | 12.8 (3.3, .) |

| | |
|---|---|
| Logrank P-value | 0.5507 |

EGFR IHC+

| | | |
|---|---|---|
| n | 12 | 20 |
| Median OS (mo) (95% Cl) | 3.4 (2.1, 4.4) | 13.5 (11.1, 15.1) |

| | |
|---|---|
| Logrank P-value | <0.001 |
| Hazard Ratio (95% Cl) | 4.9 (2.1, 11.5) |

According to the diagnostic and prognostic method of the present invention, alteration of the wild-type EGFR gene is detected. Alterations of a wild-type gene according to the present invention encompasses all forms of mutations such as insertions, inversions, deletions, and/or point mutations. Somatic mutations are those which occur only in certain tissues, e.g., in the tumor tissue, and are not inherited in the germ line. Germ line mutations can be found in any of a body's tissues. If only a single allele is somatically mutated, an early neoplastic state is indicated. However, if both alleles are mutated then a late neoplastic state is indicated. The finding of EGFR mutations is therefore a diagnostic and prognostic indicator as described herein.

The EGFR mutations found in tumor tissues may result in increased signaling activity relative to wild-type EGFR leading to a cancerous state. In order to detect the alteration of the wild-type EGFR gene a sample or biopsy of the tumor is obtained by methods well known in the art and appropriate for the particular type and location of the tumor. For instance, samples of lung cancer lesions may be obtained by resection, bronchoscopy, fine needle aspiration, bronchial brushings, or from sputum, pleural fluid or blood. Means for enriching a tissue preparation for tumor cells are known in the art. For example, the tissue may be isolated from paraffin or cryostat sections. Cancer cells may also be separated from normal cells by flow cytometry or laser capture microdissection. These as well as other techniques for separating tumor from normal cells are well known in the art. If the tumor tissue is highly contaminated with normal cells, detection of mutations is more difficult.

Detection of point mutations may be accomplished by molecular cloning of the EGFR allele (or alleles) and sequencing that allele(s) using techniques well known in the art. Alternatively, the polymerase chain reaction (PCR) can be used to amplify gene sequences directly from a genomic DNA preparation from the tumor tissue. The DNA sequence of the amplified sequences can then be determined and mutations identified therefrom. The polymerase chain reaction is well known in the art and described in Saiki et al., Science 239:487, 1988; U.S. Pat. No. 4,683,203; and U.S. Pat. No. 4,683,195.

Specific primer pairs which can be used for PCR amplification of EGFR exons 18-21 include:

<5pEGFR.ex18.out>

(SEQ ID NO: 39)

CAAATGAGCTGGCAAGTGCCGTGTC
<3pEGFR.ex18.out>

(SEQ ID NO: 40)

GAGTTTCCCAAACACTCAGTGAAAC
<5pEGFR.ex19.out>

(SEQ ID NO: 41)

GCAATATCAGCCTTAGGTGCGGCTC
<3pEGFR.ex19.out>

(SEQ ID NO: 42)

CATAGAAAGTGAACATTTAGGATGTG
<5pEGFR.ex20.out>

(SEQ ID NO: 43)

CCATGAGTACGTATTTTGAAACTC
<3pEGFR.ex20.out>

(SEQ ID NO: 44)

CATATCCCCATGGCAAACTCTTGC
<5pEGFR.ex21.out>
CTAACGTTCGCCAGCCATAAGTCC

(SEQ ID NO: 45)

<3pEGFR.ex21.out>

(SEQ ID NO: 46)

GCTGCGAGCTCACCCAGAATGTCTGG
<5pEGFR.ex18.in.m13f>

(SEQ ID NO: 47)

TGTAAACGACGGCCAGTCAAGTGCCGTGTCCTGGCACCCAAGC
<3pEGFR.ex18.in.m13r>

(SEQ ID NO: 48)

CAGGAACAGCTATGACCCCAACACTCAGTGAAACAAAGAG
<5pEGFR.ex19.in.m13f>

(SEQ ID NO: 49)

TGTAAAACGACGGCCAGTCCTTAGGTGCGGCTCCACAGC
<3pEGFR.ex19.in.m13r>

(SEQ ID NO: 50)

CAGGAAACAGCTATGACCCATTTAGGATGTGGAGATGAGC
<5pEGFR.ex20.in.m13f>

(SEQ ID NO: 51)

TGTAAAACGACGGCCAGTGAAACTCAAGATCGCATTCATGC
<3pEGFR.ex20.in.m13r>

(SEQ ID NO: 52)

CAGGAAACAGCTATGACCGCAAACTCTTGCTATCCCAGGAG
<5pEGFR.ex21.in.m13f>

(SEQ ID NO: 53)

TGTAAAACGACGGCCAGTCAGCCATAAGTCCTCGACGTGG
<3pEGFR.ex21.in.m13r>

(SEQ ID NO: 54)

CAGGAAACAGCTATGACCCATCCTCCCCTGCATGTGTTAAAC

Specific primer pairs which can be used for PCR amplification of K-Ras exon 1 include:

<5pKRAS-out>

(SEQ ID NO: 55)

TACTGGTGGAGTATTTGATAGTG
<3pKRAS-out>

(SEQ ID NO: 56)

CTGTATCAAAGAATGGTCCTG
<5pKRAS-in.m13f>

(SEQ ID NO: 57)

TGTAAAACGACGGCCAGTTAGTGTATTAACCTTATGTG
<3pKRAS-in.m13r>

(SEQ ID NO: 58)

CAGGAAACAGCTATGACCACCTCTATTGTTGGATCATATTCG

The ligase chain reaction, which is known in the art, can also be used to amplify EGFR sequences. See Wu et al., Genomics, Vol. 4, pp. 560-569 (1989). In addition, a technique known as allele specific PCR can be used. (See Ruano and Kidd, Nucleic Acids Research, Vol. 17, p. 8392, 1989.) According to this technique, primers are used which hybridize at their 3'ends to a particular EGFR mutation. If the particular EGFR mutation is not present, an amplification product is not observed. Amplification Refractory Mutation System (ARMS) can also be used as disclosed in European Patent Application Publication No. 0332435 and in Newton et al., Nucleic Acids Research, Vol. 17, p.7, 1989. Insertions and deletions of genes can also be detected by cloning, sequencing and amplification. In addition, restriction fragment length polymorphism, (RFLP) probes for the gene or surrounding marker genes can be used to score alteration of an allele or an insertion in a polymorphic fragment. Single stranded conformation polymorphism (SSCP) analysis can also be used to detect base change variants of an allele. (Orita et al., Proc. Natl. Acad. Sci. USA Vol. 86, pp. 2766-2770, 1989, and Genomics, Vol. 5, pp. 874-879, 1989). Other techniques for detecting insertions and deletions as are known in the art can be used.

Alteration of wild-type genes can also be detected on the basis of the alteration of a wild-type expression product of the gene. Such expression products include both the EGFR mRNA as well as the EGFR protein product. Point mutations may be detected by amplifying and sequencing the mRNA or via molecular cloning of cDNA made from the mRNA. The sequence of the cloned cDNA can be determined using DNA sequencing techniques which are well known in the art. The cDNA can also be sequenced via the polymerase chain reaction (PCR).

Mismatches, according to the present invention are hybridized nucleic acid duplexes which are not 100% complementary. The lack of total complementarity may be due to deletions, insertions, inversions, substitutions or frameshift mutations. Mismatch detection can be used to detect point mutations in the gene or its mRNA product. While these techniques are less sensitive than sequencing, they are simpler to perform on a large number of tumor samples. An example of a mismatch cleavage technique is the RNase protection method, which is described in detail in Winter et al., Proc. Natl. Acad. Sci. USA, Vol. 82, p. 7575, 1985 and Meyers et al., Science, Vol. 230, p. 1242, 1985. In the practice a the present invention the method involves the use of a labeled riboprobe which is complementary to the human wild-type EGFR gene coding sequence (or exons 18-21 or KDR thereof). The riboprobe and either mRNA or DNA isolated from the tumor tissue are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the EGFR mRNA or gene but can be exons 18 through 21 or the EGFR KDR or segments thereof. If the riboprobe comprises only a segment of the EGFR mRNA or gene it will be desirable to use a number of these probes to screen the whole mRNA sequence for mismatches.

In a similar manner, DNA probes can be used to detect mismatches, through enzymatic or chemical cleavage. See, e.g., Cotton et al., Proc. Natl. Acad. Sci. USA, Vol. 85, 4397, 1988; and Shenk et al., Proc. Natl. Acad. Sci. USA, Vol. 72, p. 989, 1975. Alternatively, mismatches can be detected by shifts in the electrophoretic mobility of mismatched duplexes relative to matched duplexes. See, e.g., Cariello, Human Genetics, Vol. 42, p. 726, 1988. With either riboprobes or DNA probes, the cellular mRNA or DNA which might contain a mutation can be amplified using PCR before hybridization. Changes in DNA of the EGFR gene can also be detected using Southern hybridization, especially if the changes are gross rearrangements, such as deletions and insertions.

DNA sequences of the EGFR gene which have been amplified by use of polymerase chain reaction may also be screened using allele-specific probes. These probes are nucleic acid oligomers, each of which contains a region of the EGFR gene sequence harboring a known mutation. For example, one oligomer may be about 30 nucleotides in length, corresponding to a portion of the EGFR gene sequence. By use of a battery of such allele-specific probes, PCR amplification products can be screened to identify the presence of a previously identified mutation in the EGFR gene. Hybridization of allele-specific probes with amplified EGFR sequences can be performed, for example, on a nylon filter. Hybridization to a particular probe under stringent hybridization conditions indicates the presence of the same mutation in the tumor tissue as in the allele-specific probe.

DNA sequences of the EGFR gene which have been amplified by use of polymerase chain reaction may also be screened for mutations by mass spectroscopy techniques. Amplified regions of the gene having a mutation will have a different mass spec signature than the same region without mutations.

Alteration of wild-type EGFR genes can also be detected by screening for alteration of wild-type EGFR protein. For example, monoclonal antibodies immunoreactive with EGFR can be used to screen a tissue. Lack of cognate antigen would indicate an EGFR mutation. Antibodies specific for products of mutant alleles could also be used to detect mutant EGFR gene product. Antibodies may be identified from phage display libraries. Such immunological assays can be done in any convenient format known in the art. These include Western blots, immunohistochemical assays and ELISA assays. Any means for detecting an altered EGFR protein can be used to detect alteration of wild-type EGFR genes.

Mutant EGFR genes or gene products can be detected from tumor or from other body samples such as urine, sputum or serum. The same techniques discussed above for detection of mutant EGFR genes or gene products in tumor samples can be applied to other body samples. Cancer cells are sloughed off from tumors and appear in such body samples. By screening such body samples, a simple early diagnosis can be achieved for many types of cancers. In addition, the progress of chemotherapy or radiotherapy can be monitored more easily by testing such body samples for mutant EGFR genes or gene products.

The methods of diagnosis of the present invention are applicable to any tumor in which EGFR has a role in tumorigenesis for example lung, breast, colon, glioma, bladder, liver, stomach and prostate. The diagnostic method of the present invention is useful for clinicians so that they can decide upon an appropriate course of treatment. For example, a tumor displaying alteration of both EGFR alleles might suggest a more aggressive therapeutic regimen than a tumor displaying alteration of only one EGFR allele.

The primer pairs of the present invention are useful for determination of the nucleotide sequence of a particular EGFR allele using the polymerase chain reaction. The pairs of single stranded DNA primers can be annealed to sequences within or surrounding the EGFR gene on in order to prime amplifying DNA synthesis of the EGFR gene itself. A set of these primers allows synthesis of all of the nucleotides of the EGFR exons 18 through 21. Allele specific primers can also be used. Such primers anneal only to particular EGFR mutant alleles, and thus will only amplify a product in the presence of the mutant allele as a template. In order to facilitate subsequent cloning of amplified sequences, primers may have restriction enzyme site sequences appended to their ends. Thus, all nucleotides of the primers are derived from EGFR exons 18-21 or sequences adjacent thereto except the few nucleotides necessary to form a restriction enzyme site. Such enzymes and sites are well known in the art. The primers themselves can be synthesized using techniques which are well known in the art. Generally, the primers can be made using oligonucleotide synthesizing machines which are commercially available. Design of particular primers is well within the skill of the art.

The nucleic acid probes provided by the present invention are useful for a number of purposes. They can be used in Southern hybridization to genomic DNA and in the RNase protection method for detecting point mutations already discussed above. The probes can be used to detect PCR amplification products. They may also be used to detect mismatches with the EGFR gene or mRNA using other techniques. Mismatches can be detected using either enzymes (e.g., S1 nuclease), chemicals (e.g., hydroxylamine or osmium tetroxide and piperidine), or changes in electrophoretic mobility of mismatched hybrids as compared to totally matched hybrids. These techniques are known in the art. See Novack et al., Proc. Natl. Acad. Sci. USA, Vol. 83, p. 586, 1986. Generally, the probes are complementary to EGFR exon 18-21 sequences, although generally probes to the kinase domain and segments thereof are also contemplated. An entire battery of nucleic acid probes may be used to compose a kit for detecting alteration of wild-type EGFR genes. The kit allows for hybridization to the entire exon 18-21 sequence of the EGFR gene. The probes may overlap with each other or be contiguous.

If a riboprobe is used to detect mismatches with mRNA, it is complementary to the mRNA of the EGFR gene. The riboprobe thus is an antisense probe in that it does not code for the EGFR protein because it is complementary to the sense strand. The riboprobe generally will be labeled with a radioactive, calorimetric, or fluorometric material, which can be accomplished by any means known in the art. If the riboprobe is used to detect mismatches with DNA it can be of either polarity, sense or anti-sense. Similarly, DNA probes also may be used to detect mismatches.

Predisposition to cancers can be ascertained by testing any tissue of a human for mutations of the EGFR gene. For example, a person who has inherited a germ line EGFR mutation would be prone to develop cancers. This can be determined by testing DNA from any tissue of the body. For example, blood can be drawn and DNA extracted from the cells of the blood. In addition, prenatal diagnosis can be accomplished by testing fetal cells, placental cells, or amniotic fluid for mutations of the EGFR gene. Alteration of a wild-type EGFR allele, whether for example, by point mutation or by deletion, can be detected by any of the means discussed above.

### EXAMPLES:

### Example 1

### Slide Preparation—Deparaffinization and Staining

### Submersed Sections in the Following Solutions:

Fresh xylenes (to depariffinize the sections) - 5 min
Fresh xylenes - 5 min
100% ethanol -15 sec
95% ethanol - 15 sec
70% ethanol - 15 sec
Deionized water - 15 sec
Mayer's Hematoxylin - 30 sec
Deionized water - rinse (×2) - 15 sec
70% ethanol - 15 sec
Eosin Y - 5 sec
95% ethanol - 15 sec
95% ethanol - 15 sec
100% ethanol - 15 sec
100% ethanol - 15 sec
Xylenes (to ensure dehydration of the section) - 60 sec
Air-dried for approximately 2 minutes or gently used air gun to completely remove xylenes.
The tissue was then ready for LCM.

### Example 2

### Laser Capture Microdissection and DNA Extraction

### Materials:

PixCell II LCM System
CapSure HS or CapSure Macro LCM caps
ExtractSure device (HS only)
Razor blades (factory sterile)
0.5 ml tubes
0.2 ml tubes
PicoPure DNA extraction Kit
65° C. incubator

### Procedure:

Placed CapSure cap over area of tissue to be collected
2. Lased over desired area
Lifted cap off tissue.
Dispensed 20 ul of PicoPure digest buffer with Proteinase K into 0.5 ml tube.
Placed cap with dissected material into tube to form a tight seal.
Inverted tube such that digest buffer covered cap.
Incubated at 65° C. for 24 hours.
Spun tube with cap to collect digested material in the bottom of the tube.
Transferred digest to 0.2 ml strip tube.
Inactivated Proteinase K at 95° C. for 10 minutes in a thermocycler with a heated lid.
10. Used 1-2 ul of sample in a 50 ul PCR reaction. No clean-up was necessary.

### Example 3

### PCR Amplification

### PCR Primers:

Primer pairs were designed for each exon to be sequenced (EGFR exons 18, 19, 20 and 21). Primer sequences used were as follows:

<5pEGFR.ex18.out>

(SEQ ID NO: 39)

CAAATGAGCTGGCAAGTGCCGTGTC
<3pEGFR.ex18.out>

(SEQ ID NO: 40)

GAGTTTCCCAAACACTCAGTGAAAC
<5pEGFR.ex19.out>

(SEQ ID NO: 41)

GCAATATCAGCCTTAGGTGCGGCTC
<3pEGFR.ex19.out>

(SEQ ID NO: 42)

CATAGAAAGTGAACATTTAGGATGTG
<5pEGFR.ex20.out>

(SEQ ID NO: 43)

CCATGAGTACGTATTTTGAAACTC
<3pEGFR.ex20.out>

(SEQ ID NO: 44)

CATATCCCCATGGCAAACTCTTGC
<5pEGFR.ex21.out>

(SEQ ID NO: 45)

CTAACGTTCGCCAGCCATAAGTCC
<3pEGFR.ex21.out>

(SEQ ID NO: 46)

GCTGCGAGCTCACCCAGAATGTCTGG
<5pEGFR.ex18.in.m13f>

(SEQ ID NO: 47)

TGTAACGACGGCCAGTCAAGTGCCGTGTCCTGGCACCCAAGC
<3pEGFR.ex18.in.m13r>

(SEQ ID NO: 48)

CAGGAACAGCTATGACCCCAACACTCAGTGAAACAAAGAG
<5pEGFR.ex19.in.m13f>

(SEQ ID NO: 49)

TGTAAAACGACGGCCAGTCCTTAGGTGCGGCTCCACAGC
<3pEGFR.ex19.in.m13r>

(SEQ ID NO: 50)

CAGGAAACAGCTATGACCCATTTAGGATGTGGAGATGAGC
<5pEGFR.ex20.in.m13f>

(SEQ ID NO: 51)

TGTAAAACGACGGCCAGTGAAACTCAAGATCGCATTCATGC
<3pEGFR.ex20.in.m13r>

(SEQ ID NO: 52)

CAGGAAACAGCTATGACCGCAAACTCTTGCTATCCCAGGAG
<5pEGFR.ex21.in.m13f>

(SEQ ID NO: 53)

TGTAAAACGACGGCCAGTCAGCCATAAGTCCTCGACGTGG
<3pEGFR.ex21.in.m13r>

(SEQ ID NO: 54)

CAGGAAACAGCTATGACCCATCCTCCCCTGCATGTGTTAAAC
K-Ras oligos for PCR
<5pKRAS-out>

(SEQ ID NO: 55)

TACTGGTGGAGTATTTGATAGTG
<3pKRAS-out>

(SEQ ID NO: 56)

CTGTATCAAAGAATGGTCCTG
<5pKRAS-in.m13f>

(SEQ ID NO: 57)

TGTAAAACGACGGCCAGTTAGTGTATTAACCTTATGTG
<3pKRAS-in>.m13r

(SEQ ID NO: 58)

CAGGAAACAGCTATGACCACCTCTATTGTTGGATCATATTCG

Nested amplification of the primary PCR product was performed using intron-specific primer pairs located within the primary PCR product. These nested primers pairs were tagged with M13f and M13rev sequences.

### First Round of PCR:

PCR Reaction:

| | |
|---|---|
| DNA | 0.5 to 30 ng |
| Primers | 250 nM/each outer primers |
| dNTPs | 0.2 mM each (Roche cat#1581295) |
| MgCl₂ | 1.5 mM (15 mM 10 X buffer) |
| Enzyme | 1.5 U/RX Expand High fidelity Taq (Roche cat#1759078) |
| | 50 ul reaction volume |

Thermocycler conditions:
   95° C. - 3 minutes
   94° C. - 30 seconds repeat 35 times
   58° C. - 30 seconds
   72° C. - 1 minute
   72° C. - 8 minutes
   4° C. - forever

### Second Round of PCR:

PCR Reaction:

| | |
|---|---|
| DNA | 1 ul from first round PCR reaction |
| Primers | 250 nM/each inner primers |
| dNTPs | 0.2 mM each (Roche cat#1581295) |
| MgCl₂ | 1.5 mM (15 mM 10 X buffer) |
| Enzyme | 1.5 U/RX Expand High fidelity Taq (Roche cat#1759078) |
| | 50 ul reaction volume |

Thermocycler conditions:
   95° C. - 3 minutes
   94° C. - 30 seconds repeat 30 times
   58° C. - 30 seconds
   72° C. - 1 minute
   72° C. - 8 minutes
   4° C. - forever

### Isolation of PCR Products:

PCR reaction products were run on E-Gel 2% agarose gels (Invitrogen, cat# G6018-02) for quality control. PCR products were purified directly using the Qiaquick 96 PCR purification kit (Qiagen, cat#28181) or gel purified as was necessary. For gel purification, the PCR product was excised from the E-gel and the DNA purified using Qiaquick 96 PCR purification kit with a gel extraction protocol (Qiagen, cat#28181).

### Example 4

### Sequencing

Nested sequencing primers or standard M13f and M13rev sequencing primers for tagged PCR products were used to sequence the purified PCR products. Sequences were as follows:

| | |
|---|---|
| <m13f> TGTAAAACGACGGCCAGT | (SEQ ID NO: 59) |
| <m13r> CAGGAAACAGCTATGACC | (SEQ ID NO: 60) |

Purified PCR products were diluted and cycle-sequenced using the BigDye Terminator Kit (ABI, Foster City, Calif.) according to manufacturer's instructions.

### Reaction Mix:

5 ul DNA (25-100 ng PCR product)
6 ul water
1 ul primer diluted to 0.25 OD/100 ul with water (m13f or m13r or sequence specific primer)
2 ul BigDye v3.1
6 ul Dilution Buffer (equivalent of ABI 5×Dilution Buffer)

### Cycle Sequencing:

Conditions:
96° C. - 2.5 minutes—initial denaturation
96° C. - 10 seconds
50° C. - 5 seconds
60° C. - 4 minutes
repeated for 25 to 50 total cycles

### Reaction Cleanup:

Removed unincorporated nucleotides using:
8% sephadex
500 ul in Edge BioSystem 96-well block
spin @ 750 g for 2 minutes

### Analysis:

Reaction products were electrophoresed on ABI3700 or ABI3730 sequencing instruments.

Electropherograms were analyzed for mutations using commercially available analysis programs, such as Sequencher (Gene Codes, Corp), and with custom tools.

### Example 5

### Dose Response

Human epidermal growth factor receptor (EGFR) wild-type and mutant constructs used in this study were epitope-tagged at the N-terminus with the herpes simplex virus signal sequence of gD, replacing the endogenous EGFR signal sequence (Schaefer et al. 1999 J. Biol. Chem. 274, 859-866). Cos7 cells were seeded in 12 well dishes in normal growth medium 24 hours prior to transfection. Cells were transfected with 0.25 ug per well with expression plasmid DNAs (pRK5.gD.EGFR wild-type, pRK5.gD.EGFR. L858R, or pRK5.gD.EGFR.del(E746-S752)) using LipofectAMINE 2000 following manufacturer's recommended protocol (Invitrogen). Twenty-four hours post-transfection, cells were serum starved for six hours in serum free DMEM. One hour prior to stimulation, transfected cells were preincubated with the indicated concentrations of erlotinib. Transfected cells were stimulated with 1 nM TGFα for 10 minutes. Cells were lysed directly in the wells using reducing Laemmli buffer. Receptor autophosphorylation, an index of EGFR receptor activation by growth factor stimulation, was detected by Western blotting using an HRP-conjugated anti-phosphotyrosine antibody (Oncogene Sciences, AB-4). Transfection efficiency was evaluated using an antibody specific for the gD epitope tag (5B6). Level of receptor activation was evaluated from the autoradiograms using NIH Image software. These data were then used to generate a graph from which an IC50 was calculated using a 4 parameter fit function. As illustrated by the results below, erlotinib has a greater affinity to EGFR containing mutations compared to wild-type EGFR.

| EGFR construct | inhibition (IC50) |
|---|---|
| WT EGFR-gD | 50 nM |
| L858R EGFR-gD | 20 nM |
| del(746-752) EGFR-gD | 5 nM |

We claim:

1. A method of identifying a patient nonresponsive to treatment with an EGFR inhibitor, comprising determining the presence or absence of a KRAS mutation and an EGFR in a tumor of said patient whereby the presence of both a KRAS mutation and an EGFR indicates a patient will not respond to said EGFR inhibitor treatment.

2. A method of identifying a patient nonresponsive to treatment with an EGFR inhibitor in combination with a chemotherapeutic agent, comprising determining the presence or absence of a KRAS mutation and an EGFR in a tumor of said patient whereby the presence of both a KRAS mutation and an EGFR in said tumor indicates a patient will not respond to said EGFR inhibitor treatment in combination with chemotherapy.

3. A method of determining whether a tumor will respond to treatment with an EGFR inhibitor, comprising determining in a sample of said tumor the presence of an EGFR and a mutant KRAS protein or gene whereby the presence of both an EGFR and a mutant KRAS protein or gene indicates that the tumor will not respond to treatment with an EGFR inhibitor.

4. A method of determining whether a tumor will respond to treatment with an EGFR in combination with a chemotherapeutic agent, comprising determining in a sample of said tumor the presence of an EGFR and a mutant KRAS whereby the presence of both an EGFR and a mutant KRAS protein or gene indicates that the tumor will not respond to treatment with an EGFR inhibitor.

5. The method of claim 1 wherein said KRAS mutation is an activating mutation.

6. The method of claim 1 wherein said KRAS mutation is at least one of G12C; G12A;
G12D; G12R; G12S; G12V; G13C; and G13D.

7. The method of claim 1 wherein said EGFR inhibitor is one or more of cetuximab, panitumumab, erlotinib or gefitinib.

8. The method of claim 7 wherein said EGFR inhibitor is erlotinib.

9. The method of claim 2 wherein said chemotherapeutic agent is one or more of cytarabine, fludarabine, 5-fluoro-2′-deoxyuiridine, gemcitabine, hydroxyurea, methotrexate; bleomycin, chlorambucil, cisplatin, cyclophosphamide, doxorubicin, mitoxantrone; camptothecin, topotecan; teniposide; colcemid, colchicine, paclitaxel, vinblastine vincristine or tamoxifen.

10. The method of claim 2 wherein said chemotherapeutic agent is carboplatin and/or paclitaxel.

11. The method of claim 1 wherein the EGFR is wildtype EGFR.

12. The method of claim 1 wherein the EGFR is mutated EGFR.

13. The method of claim 1 wherein the presence of EGFR is determined by immunohistochemistry.

14. The method of claim 1 wherein the presence of a KRAS mutation is determined by amplifying KRAS nucleic acid from said tumor, or a fragment thereof suspected of containing a mutation, and sequencing said amplified nucleic acid.

15. The method of claim 1 wherein the presence of a KRAS mutation is determined by amplifying KRAS nucleic acid from said tumor, or a fragment thereof suspected of containing a mutation, and comparing the electrophoretic mobility of the amplified nucleic acid to the electrophoretic mobility of corresponding wild-type KRAS nucleic acid or fragment.


SEQUENCE LISTING (60 sequences)

SEQ ID NO: 1 - protein, 1210 aa, Homo sapiens [sequence omitted]

SEQ ID NO: 2 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 3 - protein, 6 aa, Homo sapiens
KVLGSG

SEQ ID NO: 4 - DNA, 18 bp, Homo sapiens
aaagtgctgggctccggt

SEQ ID NO: 5 - DNA, 18 bp, Homo sapiens
aaagtgctgtgctccggt

SEQ ID NO: 6 - protein, 6 aa, Homo sapiens
KVLASG

SEQ ID NO: 7 - DNA, 18 bp, Homo sapiens
aaagtgctggcctccggt

SEQ ID NO: 8 - protein, 6 aa, Homo sapiens
KVLCSG

SEQ ID NO: 9 - protein, 19 aa, Homo sapiens
AIKELREATSPKANKEILD

SEQ ID NO: 10 - DNA, 57 bp, Homo sapiens
gctatcaaggaattaagagaagcaacatctccgaaagccaacaaggaaatcctcgat

SEQ ID NO: 11 - DNA, 42 bp, Homo sapiens
gctatcaaaacatctccgaaagccaacaaggaaatcctcgat

SEQ ID NO: 12 - DNA, 48 bp, Homo sapiens
gctatcaagcaaccaacatctccgaaagccaacaaggaaatcctcgat

SEQ ID NO: 13 - DNA, 39 bp, Homo sapiens
gctatcaaggaatcgaaagccaacaaggaaatcctcgat

SEQ ID NO: 14 - DNA, 33 bp, Homo sapiens
gctatcaaggaattaagagaagcaaccctcgat

SEQ ID NO: 15 - DNA, 57 bp, Homo sapiens
gctatcaaggaattaagagaagcaacatctccgaaagtcaacaaggaaatcctcgat

SEQ ID NO: 16 - DNA, 57 bp, Homo sapiens
gctatcaaggaatcaagagaagcaacatctccgaaagccaacaaggaaatcctcgat

SEQ ID NO: 17 - DNA, 57 bp, Homo sapiens
gctatcaagaaattaagagaagcaacatctccgaaagccaacaaggaaatcctcgat

SEQ ID NO: 18 - protein, 7 aa, Homo sapiens
MASVDNP

SEQ ID NO: 19 - DNA, 21 bp, Homo sapiens
atggccagcgtggacaacccc

SEQ ID NO: 20 - DNA, 27 bp, Homo sapiens
atggccatagccagcgtggacaacccc

SEQ ID NO: 21 - protein, 9 aa, Homo sapiens
MAIASVDNP

SEQ ID NO: 22 - DNA, 30 bp, Homo sapiens
atggccagcgtggccagcgtggataacccc

SEQ ID NO: 23 - protein, 10 aa, Homo sapiens
MASVASVDNP

SEQ ID NO: 24 - DNA, 21 bp, Homo sapiens
atggccatcgtggacaacccc

SEQ ID NO: 25 - protein, 7 aa, Homo sapiens
MAIVDNP

SEQ ID NO: 26 - protein, 6 aa, Homo sapiens
FGLAKL

SEQ ID NO: 27 - DNA, 18 bp, Homo sapiens
tttgggctggccaaactg

SEQ ID NO: 28 - DNA, 18 bp, Homo sapiens
tttgggcgggccaaactg

SEQ ID NO: 29 - protein, 6 aa, Homo sapiens
FGRAKL

SEQ ID NO: 30 - DNA, 18 bp, Homo sapiens
tttgggccggccaaactg

SEQ ID NO: 31 - protein, 6 aa, Homo sapiens
FGPAKL

SEQ ID NO: 32 - DNA, 10 bp, Homo sapiens
ggaattaaga

SEQ ID NO: 33 - DNA, 15 bp, Homo sapiens
ggaattaagagaagc

SEQ ID NO: 34 - DNA, 18 bp, Homo sapiens
taagagaagcaacatctc

SEQ ID NO: 35 - DNA, 18 bp, Homo sapiens
ttaagagaagcaacatct

SEQ ID NO: 36 - DNA, 12 bp, Homo sapiens
taagagaagcaa

SEQ ID NO: 37 - DNA, 24 bp, Homo sapiens
atctccgaaagccaacaaggaaat

SEQ ID NO: 38 - protein, 19 aa, Homo sapiens
KKKGPWLEEEEEAYGWLDF

SEQ ID NO: 39 - DNA, 25 bp, Homo sapiens
caaatgagctggcaagtgccgtgtc

SEQ ID NO: 40 - DNA, 25 bp, Homo sapiens
gagtttcccaaacactcagtgaaac

SEQ ID NO: 41 - DNA, 25 bp, Homo sapiens
gcaatatcagccttaggtgcggctc

SEQ ID NO: 42 - DNA, 26 bp, Homo sapiens
catagaaagtgaacatttaggatgtg

SEQ ID NO: 43 - DNA, 24 bp, Homo sapiens
ccatgagtacgtattttgaaactc

SEQ ID NO: 44 - DNA, 24 bp, Homo sapiens
catatccccatggcaaactcttgc

SEQ ID NO: 45 - DNA, 24 bp, Homo sapiens
ctaacgttcgccagccataagtcc

SEQ ID NO: 46 - DNA, 26 bp, Homo sapiens
gctgcgagctcacccagaatgtctgg

SEQ ID NO: 47 - DNA, 44 bp, Homo sapiens
tgtaaaacgacggccagtcaagtgccgtgtcctggcacccaagc

SEQ ID NO: 48 - DNA, 42 bp, Homo sapiens
caggaaacagctatgaccccaaacactcagtgaaacaaagag

SEQ ID NO: 49 - DNA, 39 bp, Homo sapiens
tgtaaaacgacggccagtccttaggtgcggctccacagc

SEQ ID NO: 50 - DNA, 40 bp, Homo sapiens
caggaaacagctatgacccatttaggatgtggagatgagc

SEQ ID NO: 51 - DNA, 41 bp, Homo sapiens
tgtaaaacgacggccagtgaaactcaagatcgcattcatgc

SEQ ID NO: 52 - DNA, 41 bp, Homo sapiens
caggaaacagctatgaccgcaaactcttgctatcccaggag

SEQ ID NO: 53 - DNA, 40 bp, Homo sapiens
tgtaaaacgacggccagtcagccataagtcctcgacgtgg

SEQ ID NO: 54 - DNA, 42 bp, Homo sapiens
caggaaacagctatgacccatcctcccctgcatgtgttaaac

SEQ ID NO: 55 - DNA, 23 bp, Homo sapiens
tactggtggagtatttgatagtg

SEQ ID NO: 56 - DNA, 21 bp, Homo sapiens
ctgtatcaaagaatggtcctg

SEQ ID NO: 57 - DNA, 38 bp, Homo sapiens
tgtaaaacgacggccagttagtgtattaaccttatgtg

SEQ ID NO: 58 - DNA, 42 bp, Homo sapiens
caggaaacagctatgaccacctctattgttggatcatattcg

SEQ ID NO: 59 - DNA, 18 bp, Homo sapiens
tgtaaaacgacggccagt

SEQ ID NO: 60 - DNA, 18 bp, Homo sapiens
caggaaacagctatgacc